(19) United States
(12) Patent Application Publication
(10) Pub. No.: US 2015/0051641 A1
(43) Pub. Date: Feb. 19, 2015

(54) PERCUTANEOUS CLOSURE DEVICE

(71) Applicant: Cook Medical Technologies LLC, Bloomington, IN (US)

(72) Inventor: Charles Leland Baxter, West Lafayette, IN (US)

(21) Appl. No.: 14/460,520

(22) Filed: Aug. 15, 2014

Related U.S. Application Data
(60) Provisional application No. 61/866,185, filed on Aug. 15, 2013.

(51) Int. Cl. A61B 17/00

(52) U.S. Cl. CPC A61B 17/0057; A61B 2017/00004; A61B 2017/00606; A61B 2017/00619
USPC 606/215

(57) ABSTRACT

Described are medical devices useful for closing a percutaneous access site, as well as methods of using same.

### REFERENCE TO RELATED APPLICATION

The present application claim the benefit of U.S. Provisional Patent Application No. 61/866,185 filed Aug. 15, 2013 which is incorporated herein by reference in its entirety.

### FIELD

The present invention relates generally to medical devices and more particularly to systems and methods for closure of percutaneous access sites.

### BACKGROUND

This disclosure concerns apparatuses and methods useful for sealing an opening in a vessel wall, such as a percutaneous access opening in the wall of a vessel. In particular, apparatus and methods are disclosed for closing and to promote healing of an opening in a vessel wall made during a medical procedure (e.g. those in which apparatus or medicaments are introduced into a vessel).

It has long been known to insert devices into bodily vessels or conduits to provide therapy or for diagnostic purposes. For example, in cardiovascular medicine, it is known to insert catheters, stents and other devices into a patient's vascular system in order to evaluate or treat the patient. In the case of percutaneous transluminal angioplasty (PTA), an opening is made through the patient's skin and into a large or relatively large blood vessel, such as the femoral or iliac arteries, and a balloon is inserted into the vessel and advanced to the location where vessel narrowing has occurred, such as by atherosclerosis. Similar procedures are used to implant stents to maintain flow through blood or other bodily vessels or ducts. In accessing the interior of a blood vessel, the surgeon or medical professional must breach the integrity of the vessel. A variety of devices (e.g. needles, guide wires, cannulae) are known to open a path into a vessel via a percutaneous opening or other approach. Additional devices or implants can be moved through such devices, or through sleeves or cannulae placed in the opening to keep it open, and into the vessel.

When the procedure is concluded, a cannula or other access device is removed from the vessel, leaving an opening in the vessel. If the vessel puncture is not adequately closed, a subcutaneous hematoma will form. The medical professional must therefore take steps to close the opening in the vessel. In some cases, the opening may be sutured closed, but such action can be very difficult in close quarters, and many vessel-accessing procedures are intended to be minimally-invasive to reduce tissue damage. It is also known to apply constant, firm external pressure to the opening in the vessel, particularly if it is a blood vessel, to allow the body's natural coagulation and healing processes to work. In cases in which angioplasty or similar treatment has taken place, however, commonly an anticoagulant has been administered to the patient, making natural closing of the opening in the vessel wall a longer or more difficult process. Maintaining physical pressure on a relatively large blood vessel for a time period sufficient for natural closure also presents at least inconvenience and discomfort to the patient in having to remain still and submit to that pressure, and there is the risk that too much pressure can damage the vessel or tissues that rely on continued circulation.

Devices have been created for closing a punctured blood vessel that are designed to block the opening and/or soak up fluids that escape the vessel, or are present in the opening through the skin leading to the vessel. Such devices have, however, proven unsatisfactory in many respects. For example, some devices leave permanent artifacts in the vessel wall or the wound track which could potentially interfere with attempts at reintervention or risk infection. In another aspect, some devices require the physician to place the device prior to the primary procedure, limiting the physicians closure options should a larger or smaller puncture be required. Needs therefore exist for improved and/or alternate devices and systems for closure of percutaneous access sites.

### SUMMARY

In certain aspects, the present invention provides unique medical devices for closure of percutaneous access sites. In accordance with some forms of the invention, such medical devices are configured to secure bioresorbable material against a vessel wall to close percutaneous access sites. Accordingly, in one embodiment, the present invention provides a medical device that includes a first bioresorbable sheet, a second bioresorbable sheet, an anchor member, and a locking member. The anchor member has a shaft and a series of radially disposed fingers configured to secure the first sheet against the interior vessel wall adjacent to the puncture site. The locking member has an opening which is configured to attach to the shaft of the anchor member, the locking member further includes a series of radially disposed fingers configured to secure the second sheet against the exterior vessel wall adjacent to the puncture site. In one aspect, the radially disposed fingers are independent of one another and are independently flexible. In certain modes of practicing the invention, the radially disposed fingers are cuneate in form and/or concave with respect to the vessel surface.

In one form, the anchor member and/or the locking member are composed of a resiliently deformable polymer. In one aspect, the anchor member and/or the locking member are composed of a bioresorbable material. In one embodiment, the anchor member and/or the locking member are composed of poly lactic-co-glycolic acid.

In one form, the first bioresorbable sheet and/or the second bioresorbable sheet are composed of extracellular matrix material. In one embodiment, the first bioresorbable sheet and/or the second bioresorbable sheet are composed of a remodelable material.

Certain inventive variants further include a series of protrusions on the shaft to which the locking member is adapted to attach. In one form, the shaft contains a compression limiting element. In one embodiment, the compression limiting element comprises a portion of the shaft with a larger diameter than the opening in the locking member, which prevents the locking member from exerting excessive pressure on the vein. In one form, the disclosed device shaft further includes a retention element. The device may also include a filament extending from the shaft.

In one embodiment, the present disclosure describes a method for closing a puncture in the wall of a blood vessel. The described method includes the steps of: inserting an anchor member and a first bioresorbable sheet through a puncture in the vessel wall, and attaching a locking member with a second bioresorbable sheet to the shaft of the anchor member external to the punctured vessel. In certain modes of practicing the invention, a retention element is included in the form of an elongated shaft. The retention element is used to secure the implanted anchor at the puncture site before deployment of the locking member.

In yet another embodiment the present disclosure describes a device for closing a puncture in the wall of a body vessel. The device includes an anchor member which has a series of radially disposed fingers extending from a shaft. In accordance with certain inventive variants the anchor member is self expandable from a first position in which the fingers are collapsed along the shaft for delivery through the puncture site, to a second position in which the fingers are extended transverse to the axis of the shaft. The device further includes a locking member which defines an opening configured to receive and attach to the anchor member shaft, the opening is surrounded by a series of radially disposed fingers extending outward from the opening. In one aspect, the radially disposed fingers on each the anchor and the lock are independent of one another and are independently flexible. In certain modes of practicing the invention, the radially disposed fingers are cuneate in form and/or concave with respect to the vessel surface.

In one form, the anchor member and/or the locking member are composed of a resiliently deformable polymer. In one aspect, the anchor member and/or the locking member are composed of a bioresorbable material. In one embodiment, the anchor member and/or the locking member are composed of poly lactic-co-glycolic acid.

In one embodiment the disclosed device includes a first bioresorbable sheet and second bioresorbable sheet. The first sheet contains a first opening configured to fit over the shaft such that in use the sheet is secured between the internal vessel wall and the fingers of the anchor member. The second sheet contains a second opening also configured to fit over the shaft such that in use the second sheet is secured between the external vessel wall and the fingers of the locking member. In further inventive variants, the first and second sheets are composed of a remodelable material. In yet another aspect the first sheet may be attached to the anchor member and/or the second sheet may be attached to the locking member.

Certain inventive variants further include a series of protrusions on the shaft to which the opening of the locking member is adapted to attach. In one form, the shaft contains a compression limiting element.

Further forms, objects, features, aspects, benefits, advantages, and embodiments of the present invention will become apparent from a detailed description and drawings provided herewith.

### BRIEF DESCRIPTION OF THE DRAWINGS

FIG. 1 is a perspective view of an embodiment of the disclosed device, showing an anchor member engaged with a locking member.

FIG. 2a is a perspective view of one embodiment of a locking member.

FIG. 2b is a perspective view of one embodiment of an anchor member.

FIG. 3 a top down view of one embodiment of a bioresorbable sheet FIG. 4a is a cross-sectional view of one embodiment of the disclosed device engaged with a vessel wall.

FIG. 4b is a cross-sectional view of another exemplary embodiment of the disclosed device engaged with a vessel wall.

FIG. 5 is a cross-sectional view of one embodiment of the device within a delivery tube.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, and alterations and modifications in the illustrated device, and further applications of the principles of the disclosure as illustrated therein are herein contemplated as would normally occur to one skilled in the art to which the disclosure relates.

With respect to the specification and claims, it should be noted that the singular forms “a”, “an”, “the”, and the like include plural referents unless expressly discussed otherwise. As an illustration, references to “a device” or “the device” include one or more of such devices and equivalents thereof. It also should be noted that directional terms, such as “up”, “down”, “top”, “bottom”, and the like, are used herein solely for the convenience of the reader in order to aid in the reader's understanding of the illustrated embodiments, and it is not the intent that the use of these directional terms in any manner limit the described, illustrated, and/or claimed features to a specific direction and/or orientation.

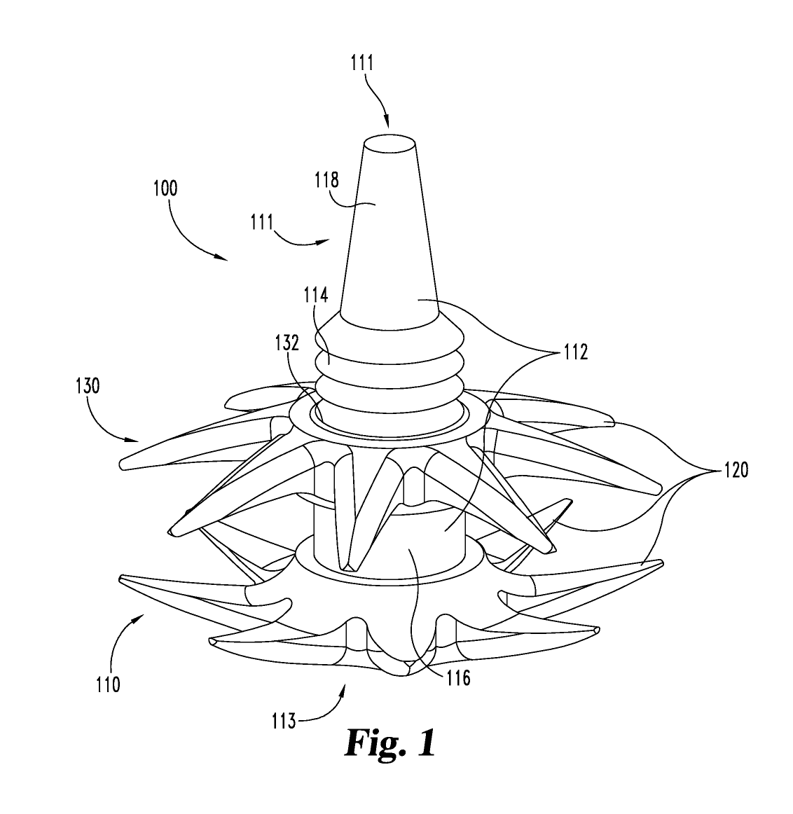

Referring now to an exemplary embodiment illustrated in FIG. 1 a device 100 for closing an opening in a wall of a vessel conduit or other bodily cavity comprises a locking member 130 and an anchor member 110. In accordance with certain inventive variants, locking member 130 contains an opening 132 and a series of radially disposed fingers 120. In certain embodiments anchor member 110 includes a shaft 112 having a proximal end 111, and a distal end 113. In certain forms distal end 113 supports a series of radially disposed fingers 120. In accordance with certain inventive variants, anchor member 110 may also include retention element 118, and/or compression limiting element 116. In certain aspects, shaft 112 may also include one or more protrusions 114. In some forms, opening 132 is sized and configured to receive shaft 112. Additionally, in some embodiments, protrusions 114 are sized and configured to secure locking member 130 to anchor member 110.

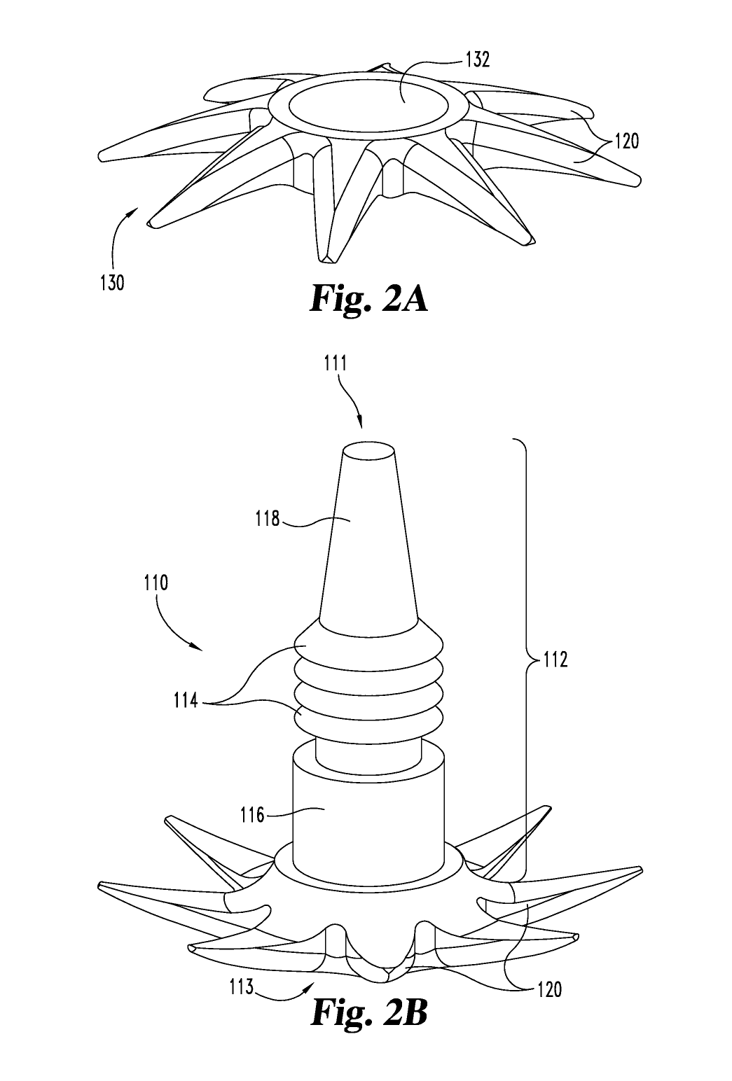

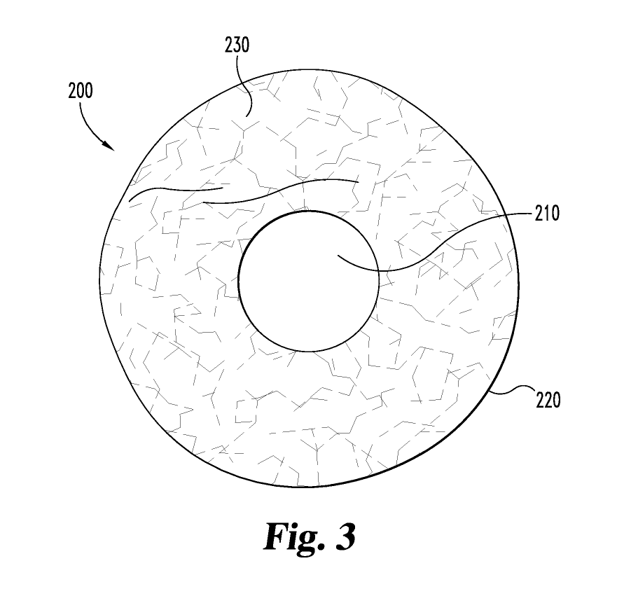

Turning now to FIG. 2, FIG. 2A shows locking member 130 with opening 132 and a series of radially disposed fingers 120. FIG. 2B shows anchor member 110 with shaft 112 and a series of radially disposed fingers 120. In certain embodiments, shaft 112 may include retention element 118, and/or protrusions 114, and/or compression limiting element 116.

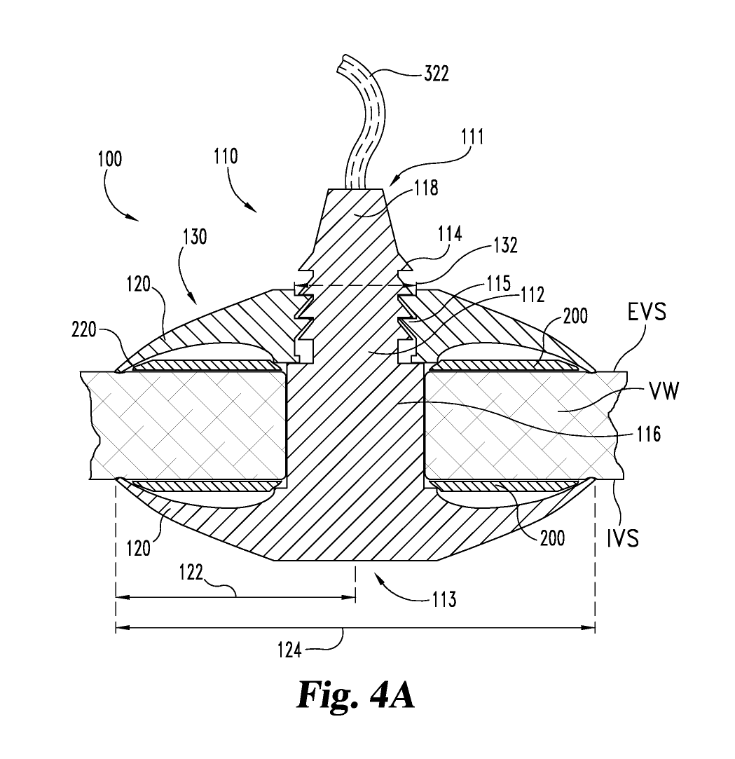

With reference now to FIG. 3 a bioresorbable sheet 200 is composed primarily of resorbable material 230 having outer edge 220. In certain embodiments, bioresorbable sheet 200 contains hole 210 sized and configured to receive shaft 112. In certain embodiments, the resorbable material is composed of one or more layers of resorbable material, for example extracellular matrix material. Bioresorbable sheet 200 is shown substantially circular. It is envisioned that bioresorbable sheet 200, may be provided in alternative formats including but not limited to: square, hexagonal, ovoid, triangular, star-shape or rectangular.

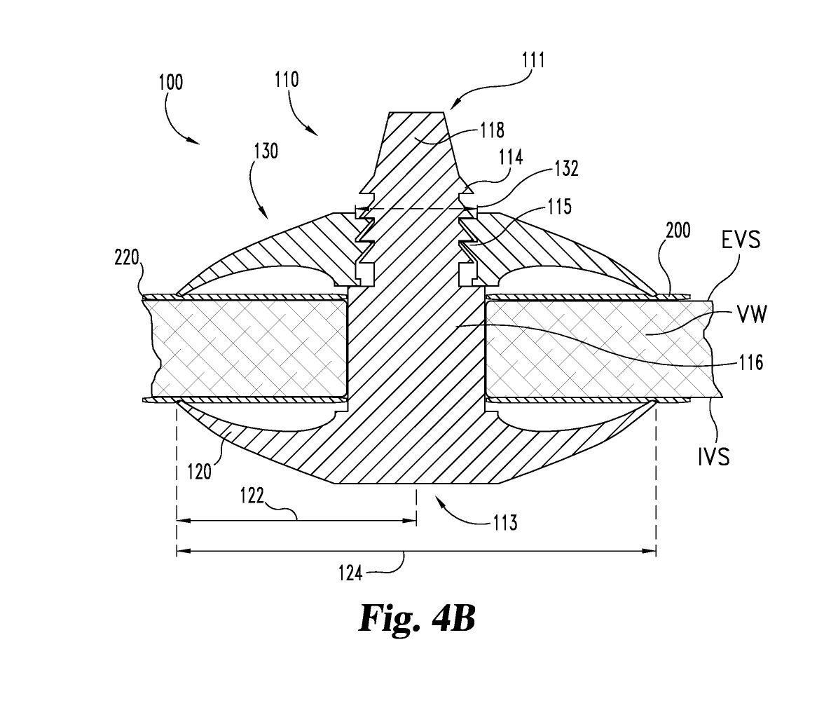

With reference to FIG. 4, FIG. 4a is a cross-sectional view of one embodiment of the disclosed device 100 engaged with vascular wall VW having an internal vascular surface IVS and external vascular surface EVS. In the illustrated embodiment, anchor member 110 includes radially disposed fingers 120 and shaft 112. Shaft 112 is configured to extend through a puncture in the vessel wall and may include retention element 118, and/or protrusions 114, and/or compression limiting element 116. In some forms, locking member 130 includes opening 132 and a series of radially disposed fingers 120. Opening 132 may include one or more locking protrusions 115 configured to interact with protrusions 114. In accordance with certain forms of the disclosure, protrusions 114 are generally frustoconical interlocking with inverted frustoconical locking protrusions 115. This frustoconical shape is provided for example only and other locking geometries and methods apparent to one of ordinary skill in the art are envisioned. As illustrated in FIG. 4, bioresorbable sheet 200 may be held against external vascular wall EVS and/or internal vascular wall IVS by a series of radially disposed fingers 120 of anchor member 110 and/or locking member 130.

Device 100 is designed to hold bioresorbable sheet 200 substantially about the puncture site promoting tissue ingrowth and healing of the site. According to certain forms of the invention, radially disposed fingers 120 are configured to be resiliently deformable to allow for insertion through the puncture site, for example via a cannulated device. In certain forms, radially disposed fingers 120 are configured to be independently flexible which allows the fingers to apply pressure to irregular surfaces for example those caused by: the curvature of the vascular wall, calcification, or other debris that may be present. Exemplary configurations include radially disposed fingers 120 which are generally concave with respect to the vessel wall. Radially disposed fingers 120 may also have cuneate or wedge-like geometry. In some forms, radially disposed fingers 120 are connected to device 100 only at the base of each finger. Other forms such as rectangular, parallelepiped, pyramidal, conical and/or cylindrical are provided as additional non-limiting examples. It is also envisioned that radially disposed fingers 120 of locking member 130 may be deployed rotated with respect to radially disposed fingers 120 of anchor member 110 creating an interdigitation and increased hold on vascular wall VW.

Device 100 is sized and configured to close punctures in vessel walls. Finger length 122, measured from the center of the device to the tip of the finger, depends on the size of the puncture to be closed. In certain embodiments, the diameter 124 of device 100 should be about 10-20% larger than the maximum dimension of the puncture to be closed. Diameter 124 is defined by the arc made by the tips of radially disposed fingers 120. Percutaneous access sites typically assume an oval shape because the tissue deforms or tears more easily along the longitudinal axis of the vessel. For example, 12-14 Fr sheaths generally create an ovoid vessel puncture of about 3 mm by about 5 mm. In this example, the desired finger length 122 would be about 3 mm, with a device diameter 124 about 6 mm.

In accordance with certain embodiments bioresorbable sheet 200 is sized and configured to fit within device diameter 124. In some forms, as illustrated in FIG. 4a, radially disposed fingers 120 may contact the vessel wall beyond outer edge 220 of bioresorbable disk 200. In other embodiments, such as that illustrated in FIG. 4b, bioresorbable sheet 200 is configured to extend beyond the device diameter 124. In some forms, radially disposed fingers 120 may contact the bioresorbable sheet 200 causing it to push against the vascular wall. In yet other embodiments the bioresorbable sheet is configured to extend about equidistant with the diameter 124 of the device. In other embodiments bioresorbable sheet 200 is configured such that portions of the sheet extend beyond the device diameter 124 and portions of the sheet are within or extend about equidistant with the device diameter 124.

Certain embodiments of the disclosed device include a compression limiting element 116. The compression limiting element 116 is configured to stop or limit the advancement of locking member 130 along shaft 112 to prevent over-compression and vascular damage. Compression limiting element 116 may, for example, have a larger diameter than opening 132 preventing passage of locking member 130. In another example, compression limiting element 116 has a substantially smooth surface free of protrusions 114 such that locking member 130 cannot lock into place.

In certain embodiments device 100 includes retention element 118. In some forms retention element 118 comprises an elongate portion of shaft 112. In certain embodiments, retention element 118 is configured to extend through the puncture opening after insertion of anchor member 110, so as to allow the physician a surface with which to retain the implanted anchor and prevent loss of the anchor into the vessel prior to attachment of the locking member 130. In some forms, retention member 118 includes a filament 322 configured to allow the physician to apply tension to anchor member 110 before locking member 130 is attached. In some forms filament 322 is configured to guide locking member 130 to anchor member 110, for example by passing through opening 132.

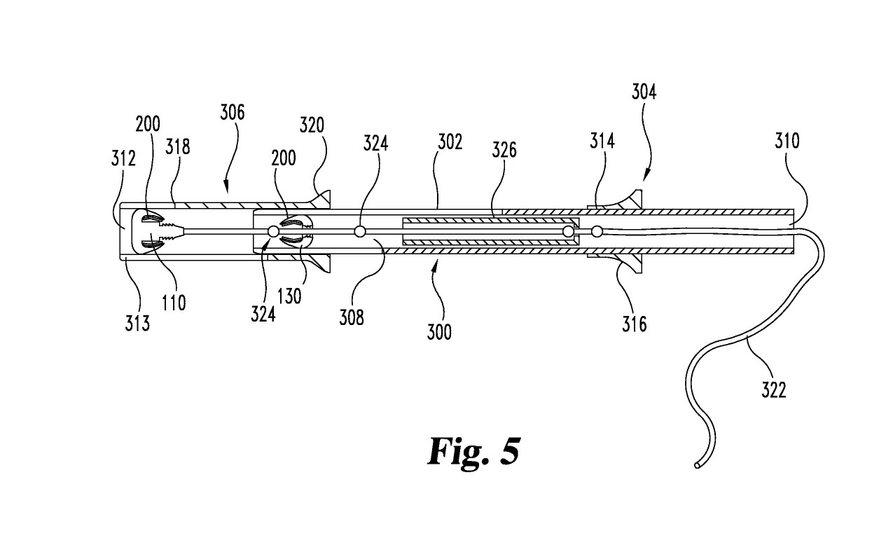

With reference now to FIG. 5 a delivery apparatus, as described in U.S. patent application Ser. No. 13/303,707 entitled “Devices and Methods for Sealing Bodily Openings” (published as 2012/0116447 A1) which is hereby incorporated by reference, may be used. The described delivery apparatus herein discussed in meant for example only, in no way is the disclosed closure device limited to delivery by the discussed apparatus. Delivery tube 300 has a main body portion 302, a proximal or upper boss 304 and a slidable distal or lower boss 306. Body 302 is a cylindrical tube in this embodiment, with a constant-diameter lumen 308 extending throughout between a proximal opening 310 and a distal opening 312 at an end 313. Upper boss 304 is fixed with respect to body 302 or made separately and fixed to body 302, as by gluing or welding. In the illustrated embodiment, upper boss 304 has a substantially cylindrical part 314 that flares outward into a widened part 316, to give a shape akin to the bell of a trumpet. Lower boss 306 has a similar shape, with a cylindrical part 318 and a flared part 320, and is slidable between distal opening 312 and upper boss 304. Flared part 320 of lower boss 306 allows cylindrical part 314 of upper boss 304 to enter it and widened part 316 of upper boss 304 engages the flared part 320 of lower boss 306 to stop travel of boss 304 along body portion 302 of delivery tube 300. Lower boss 306 of delivery tube 300 is sized to be able to be at least partially inserted into a sheath having access to patient vasculature.

In certain embodiments filament 322 extends from anchor member 110, filament 322 may for example be embedded in retention element 118. In some forms filament 322 extends from anchor member 110 through lumen 308 and out of delivery tube 300 through proximal opening 310. In some embodiments filament 322 extends through opening 132 of locking member 130. Filament 322 may also extend through a pusher 326 contained in upper boss 304. As disclosed filament 322 may additionally contain stop elements 324.

To prepare delivery tube 300 for use, anchor member 110 and locking member 130, each with a bioresorbable sheet 200, are assembled with delivery tube 300. As noted above, filament 322, and pusher 326 may also be included. Alternatively, anchor member 110, locking member 130, and/or bioresorbable sheets 200 may optionally be advanced down the delivery tube 300 after insertion into a sheath having access to patient vasculature. Anchor member 110 is compressed, such that the radially disposed fingers 120 are folded inwards towards shaft 112, and inserted into lower boss 306. In its compressed state, anchor member 110 is under stress and tries to regan its unstressed state, and so it is in affirmative engagement with the inside of boss 306. Likewise, locking member 130 is compressed, such that the radially disposed fingers 120 are folded inwards towards opening 132, and inserted into upper boss 304. In its compressed state, locking member 130 is under stress and tries to regain its unstressed state, and so it is in affirmative engagement with the inside of boss 304.

The use of delivery tube 300 is described in the reference incorporated above. In general the delivery tube is inserted into a sheath, cannula, dilator, or other access device or portal having access to patient vasculature. Slidably advancing upper boss 304 relative to lower boss 306 causes upper boss to engage anchor member 110, and a bioresorbable sheet 200, pushing both out of distal opening 312. If distal opening 312 is not located within the patient vessel upper boss 304 can be further advanced to push anchor member 110 and bioresorbable sheet 200 through the access device and into the vessel. Tension on filament 322 may be used to retain anchor member 110 within the vessel and to pull anchor member 110 and bioresorbable sheet 200 against the vessel wall. Locking member 130 is deployed by advancing pusher 326 within upper boss 304 against locking member 130. In certain embodiments filament 322 passes through opening 132 and pusher 326, this configuration allows filament 322 to guide opening 132 to shaft 112. Other embodiments may utilize the diameter of the inner lumen 308 to align opening 132 and shaft 112. Locking member 130 is advanced until protrusions 114, or other locking means, engage with opening 132 of locking member 130.

In certain embodiments anchor member 110, locking member 130, and/or filament 322 comprise a biodegradable material so that they are resorbed by the body after the vessel with which they are used has been repaired. In some forms the biodegradable materials is a biodegradable polymer. In accordance with some embodiments the polymer is resiliently deformable and/or elastomeric. In certain embodiments the material exhibits a durometer of greater than about 80 shore A. In certain embodiments the material is selected so that anchor 110 and locking 130 members degrade in about one month.

In certain embodiments one or more device components, for example bioresorbable sheet 200, will be comprised of a remodelable material. Particular advantage can be provided by devices that incorporate a remodelable collagenous material. Such remodelable collagenous materials, whether reconstituted or naturally derived, can be provided, for example by collagenous materials isolated from a warm-blooded vertebrate, especially from a mammal. Such isolated collagenous material can be processed so as to have remodelable angiogenic properties and promote cellular invasion and ingrowth. Remodelable materials may be used in this context to promote cellular growth on around and/or in bodily regions in which inventive devices are implanted or engrafted.

Suitable remodelable materials can be provided by collagenous extracellular matrix (ECM) materials possessing biotropic properties. For example, suitable collagenous materials include ECM materials such as those comprising submucosa, renal capsule membrane, dermal collagen, dura mater, pericardium, fascia lata, serosa, peritoneum or basement membrane layers, including liver basement membrane. Suitable submucosa materials for these purposes include, for instance, intestinal submucosa including small intestinal submucosa, stomach submucosa, urinary bladder submucosa, and uterine submucosa. Collagenous matrices comprising submucosa (potentially along with other associated tissues) useful in the present invention can be obtained by harvesting such tissue sources and delaminating the submucosa-containing matrix from smooth muscle layers, mucosal layers, and/or other layers occurring in the tissue source. For additional information as to some of the materials useful in the present invention, and their isolation and treatment, reference can be made, for example, to U.S. Pat. Nos. 4,902,508, 5,554,389, 5,993,844, 6,206,931, and 6,099,567.

Submucosa-containing or other ECM tissue used in the invention is preferably highly purified, for example, as described in U.S. Pat. No. 6,206,931 to Cook et al. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of any ECM tissue used in the present invention.

A typical layer thickness for an isolated submucosa or other ECM tissue layer used in the invention ranges from about 50 to about 250 microns when fully hydrated, more typically from about 50 to about 200 microns when fully hydrated, although isolated layers having other thicknesses may also be obtained and used. These layer thicknesses may vary with the type and age of the animal used as the tissue source. As well, these layer thicknesses may vary with the source of the tissue obtained from the animal source.

The ECM tissue material utilized desirably retains a structural microarchitecture from the source tissue, including structural fiber proteins such as collagen and/or elastin that are non-randomly oriented. Such non-random collagen and/or other structural protein fibers can in certain embodiments provide an ECM tissue that is non-isotropic in regard to tensile strength, thus having a tensile strength in one direction that differs from the tensile strength in at least one other direction.

The ECM tissue material may include one or more bioactive agents. Suitable bioactive agents may include one or more bioactive agents native to the source of the ECM tissue material. For example, a submucosa or other remodelable ECM tissue material may retain one or more growth factors such as but not limited to basic fibroblast growth factor (FGF-2), transforming growth factor beta (TGF-beta), epidermal growth factor (EGF), cartilage derived growth factor (CDGF), and/or platelet derived growth factor (PDGF). As well, submucosa or other ECM materials when used in the invention may retain other native bioactive agents such as but not limited to proteins, glycoproteins, proteoglycans, and glycosaminoglycans. For example, ECM materials may include heparin, heparin sulfate, hyaluronic acid, fibronectin, cytokines, and the like. Thus, generally speaking, a submucosa or other ECM material may retain one or more bioactive components that induce, directly or indirectly, a cellular response such as a change in cell morphology, proliferation, growth, and protein or gene expression.

Submucosa-containing or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with appropriate staining. Such non-collagenous solids can constitute a significant percentage of the dry weight of the ECM material in certain inventive embodiments, for example at least about 1%, at least about 3%, and at least about 5% by weight in various embodiments of the invention.

The submucosa-containing or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the formation of new blood vessels into the materials. Methods for measuring in vivo angiogenesis in response to biomaterial implantation have recently been developed. For example, one such method uses a subcutaneous implant model to determine the angiogenic character of a material. See, C. Heeschen et al., Nature Medicine 7 (2001), No. 7, 833-839. When combined with a fluorescence microangiography technique, this model can provide both quantitative and qualitative measures of angiogenesis into biomaterials. C. Johnson et al., Circulation Research 94 (2004), No. 2, 262-268.

Further, in addition or as an alternative to the inclusion of such native bioactive components, non-native bioactive components such as those synthetically produced by recombinant technology or other methods (e.g., genetic material such as DNA), may be incorporated into an ECM material. These non-native bioactive components may be naturally-derived or recombinantly produced proteins that correspond to those natively occurring in an ECM tissue, but perhaps of a different species. These non-native bioactive components may also be drug substances. Illustrative drug substances that may be added to materials include, for example, anti-clotting agents, e.g. heparin, antibiotics, anti-inflammatory agents, thrombus-promoting substances such as blood clotting factors, e.g., thrombin, fibrinogen, and the like, and anti-proliferative agents, e.g. taxol derivatives such as paclitaxel. Such non-native bioactive components can be incorporated into and/or onto ECM material in any suitable manner, for example, by surface treatment (e.g., spraying) and/or impregnation (e.g., soaking), just to name a few. Also, these substances may be applied to the ECM material in a premanufactured step, immediately prior to the procedure (e.g., by soaking the material in a solution containing a suitable antibiotic such as cefazolin), or during or after engraftment of the material in the patient.

Inventive devices can incorporate xenograft material (i.e., cross-species material, such as tissue material from a non-human donor to a human recipient), allograft material (i.e., interspecies material, with tissue material from a donor of the same species as the recipient), and/or autograft material (i.e., where the donor and the recipient are the same individual). Further, any exogenous bioactive substances incorporated into an ECM material may be from the same species of animal from which the ECM material was derived (e.g. autologous or allogenic relative to the ECM material) or may be from a different species from the ECM material source (xenogenic relative to the ECM material). In certain embodiments, ECM material will be xenogenic relative to the patient receiving the graft, and any added exogenous material(s) will be from the same species (e.g. autologous or allogenic) as the patient receiving the graft. Illustratively, human patients may be treated with xenogenic ECM materials (e.g. porcine-, bovine- or ovine-derived) that have been modified with exogenous human material(s) as described herein, those exogenous materials being naturally derived and/or recombinantly produced.

In certain forms, inventive devices, including for example sheet component(s) 200 thereof, include a material receptive to tissue ingrowth. Upon deployment of such devices in accordance with the present invention, cells from the patient can infiltrate the material, leading to, for example, new tissue growth on, around, and/or within the device. In some embodiments, the device comprises a remodelable material. In these embodiments, the remodelable material promotes and/or facilitates the formation of new tissue, and is capable of being broken down and replaced by new tissue. In certain embodiments, the implanted device contracts in response to ingrowth of patient tissue. Remodelable ECM materials having a relatively more open matrix structure (i.e., higher porosity) are capable of exhibiting different material properties than those having a relatively more closed or collapsed matrix structure. For example, an ECM material having a relatively more open matrix structure is generally softer and more readily compliant to an implant site than one having a relatively more closed matrix structure. Also, the rate and amount of tissue growth in and/or around a remodelable material can be influenced by a number of factors, including the amount of open space available in the material's matrix structure for the infusion and support of a patient's tissue-forming components, such as fibroblasts. Therefore, a more open matrix structure can provide for quicker, and potentially more, growth of patient tissue in and/or around the remodelable material, which in turn, can lead to quicker remodeling of the material by patient tissue.

In this regard, any ECM material or bioresorbable material can have a level or degree of porosity. In certain embodiments, the porosity of a layer of ECM material is lowered by drying the material under compression. In general, compressing a pliable open matrix material, such as a pliable ECM material, increases the material's bulk density and decreases the material's porosity by decreasing the size of the voids in the open matrix. As is the case in certain aspects of the invention, when such a material is dried while being compressed, particularly under vacuum pressing conditions, the open matrix structure can become somewhat fixed in this relatively higher bulk density, lower porosity state (i.e., in a relatively more collapsed state). It should be noted that different compressing and drying techniques and/or methods, including different degrees of compressing and drying, can be designed through routine experimentation so as to allow for a material layer having an optimal degree of material bulk density and/or porosity for a particular application or procedure.

It is sometimes advantageous to perform drying operations under relatively mild temperature exposure conditions that minimize deleterious effects upon the ECM materials of the invention, for example native collagen structures and potentially bioactive substances present. Thus, drying operations conducted with no or substantially no duration of exposure to temperatures above human body temperature or slightly higher, say, no higher than about 38° C., will preferably be used in some forms of the present invention. These include, for example, vacuum pressing operations at less than about 38° C., forced air drying at less than about 38° C., or either of these processes with no active heating—at about room temperature (about 25° C.) or with cooling. Relatively low temperature conditions also, of course, include lyophilization conditions.

In additional embodiments, bioresorbable sheet materials (e.g. sheet(s) 200) useful in the invention can be made from ECM's or other collagenous materials that have been subjected to processes that expand the materials. In certain forms, such expanded materials can be formed by the controlled contact of an ECM material with one or more alkaline substances until the material expands, and the isolation of the expanded material. Illustratively, the contacting can be sufficient to expand the ECM material to at least 120% of (i.e. 1.2 times) its original bulk volume, or in some forms to at least about two times its original volume. Thereafter, the expanded material can optionally be isolated from the alkaline medium, e.g. by neutralization and/or rinsing. The collected, expanded material can be used in any suitable manner in the preparation of a graft device. Illustratively, the expanded material can be enriched with bioactive components, dried, and/or molded, etc., in the formation of a graft construct of a desired shape or configuration. In certain embodiments, a dried graft construct formed with the expanded ECM material can be highly compressible (or expandable) such that the material can be compressed for delivery, such as from within the lumen of a cannulated delivery device, and thereafter expand upon deployment from the device so as to become anchored within a patient and/or cause closure of a bodily segment within the patient.

Expanded collagenous or ECM materials can be formed by the controlled contact of a collagenous or ECM material with an aqueous solution or other medium containing sodium hydroxide. Alkaline treatment of the material can cause changes in the physical structure of the material that in turn cause it to expand. Such changes may include denaturation of the collagen in the material. In certain embodiments, it is preferred to expand the material to at least about three, at least about four, at least about 5, or at least about 6 or even more times its original bulk volume. The magnitude of the expansion is related to several factors, including for instance the concentration or pH of the alkaline medium, exposure time, and temperature used in the treatment of the material to be expanded.

ECM materials that can be processed to make expanded materials can include any of those disclosed herein or other suitable ECM's. Typically such ECM materials will include a network of collagen fibrils having naturally-occurring intramolecular cross links and naturally-occurring intermolecular cross links. Upon expansion processing as described herein, the naturally-occurring intramolecular cross links and naturally-occurring intermolecular cross links can be retained in the processed collagenous matrix material sufficiently to maintain the collagenous matrix material as an intact collagenous sheet material; however, collagen fibrils in the collagenous sheet material can be denatured, and the collagenous sheet material can have an alkaline-processed thickness that is greater than the thickness of the starting material, for example at least 120% of the original thickness, or at least twice the original thickness.

Illustratively, the concentration of the alkaline substance for treatment of the remodelable material can be in the range of about 0.5 to about 2 M, with a concentration of about 1 M being more preferable. Additionally, the pH of the alkaline substance can in certain embodiments range from about 8 to about 14. In preferred aspects, the alkaline substance will have a pH of from about 10 to about 14, and most preferably of from about 12 to about 14.

In addition to concentration and pH, other factors such as temperature and exposure time will contribute to the extent of expansion, as discussed above. In this respect, in certain variants, the exposure of the collagenous material to the alkaline substance is performed at a temperature of about 4 to about 45° C. In preferred embodiments, the exposure is performed at a temperature of about 25 to about 40° C., with 37° C. being most preferred. Moreover, the exposure time can range from at least about one minute up to about 5 hours or more. In some embodiments, the exposure time is about 1 to about 2 hours. In a particularly preferred embodiment, the collagenous material is exposed to a 1 M solution of NaOH having a pH of 14 at a temperature of about 37° C. for about 1.5 to 2 hours. Such treatment results in collagen denaturation and a substantial expansion of the remodelable material. Denaturation of the collagen matrix of the material can be observed as a change in the collagen packing characteristics of the material, for example a substantial disruption of a tightly bound collagenous network of the starting material. A non-expanded ECM or other collagenous material can have a tightly bound collagenous network presenting a substantially uniform, continuous surface when viewed by the naked eye or under moderate magnification, e.g. 100× magnification. Conversely, an expanded collagenous material can have a surface that is quite different, in that the surface is not continuous but rather presents collagen strands or bundles in many regions that are separated by substantial gaps in material between the strands or bundles when viewed under the same magnification, e.g. about 100×. Consequently, an expanded collagenous material typically appears more porous than a corresponding non-expanded collagenous material. Moreover, in many instances, the expanded collagenous material can be demonstrated as having increased porosity, e.g. by measuring for an increased permeability to water or other fluid passage as compared to the non-treated starting material. The more foamy and porous structure of an expanded ECM or other collagenous material can allow the material to be cast or otherwise prepared into a variety of three-dimensionally stable shapes for use in the preparation of medical materials and devices. It can further allow for the preparation of constructs that are highly compressible and which expand after compression. Such properties can be useful, for example, when the prepared graft construct is to be compressed and loaded into a deployment device (e.g. a lumen thereof) for delivery into a patient, and thereafter deployed to expand at the implant site.

After such alkaline treatments, the material can be isolated from the alkaline medium and processed for further use. Illustratively, the collected material can be neutralized and/or rinsed with water to remove the alkalinity from the material, prior to further processing of the material to form a graft construct.

A starting ECM material (i.e., prior to treatment with the alkaline substance) can optionally include a variety of bioactive or other non-collagenous components including, for example, growth factors, glycoproteins, glycosaminoglycans, proteoglycans, nucleic acids, and lipids. Treating the material with an alkaline substance may reduce the quantity of one, some or all of such non-collagenous components contained within the material. In certain embodiments, controlled treatment of the remodelable material with an alkaline substance will be sufficient to create a remodelable collagenous material which is substantially devoid of nucleic acids and lipids, and potentially also of growth factors, glycoproteins, glycosaminoglycans, and proteoglycans.

In certain embodiments, one or more bioactive components, exogenous or endogenous, for example, similar to those removed from an expanded material during alkaline processing, can be returned to the material. For example, an expanded material can include a collagenous material which has been depleted of nucleic acids and lipids, but which has been replenished with growth factors, glycoproteins, glycosaminoglycans, and/or proteoglycans. These bioactive components can be returned to the material by any suitable method. For instance, in certain forms a tissue extract, such as is discussed in U.S. Pat. No. 6,375,989 which is hereby incorporated herein by reference in its entirety, containing these components can be prepared and applied to an expanded collagenous material. In one embodiment, the expanded collagenous material can be incubated in a tissue extract for a sufficient time to allow bioactive components contained therein to associate with the expanded collagenous material. The tissue extract may, for example, be obtained from non-expanded collagenous tissue of the same type used to prepare the expanded material. Other means for returning or introducing bioactive components to an expanded remodelable collagenous material include spraying, impregnating, dipping, etc. as known in the art. By way of example, an expanded collagenous material may be modified by the addition of one or more growth factors such as basic fibroblast growth factor (FGF-2), transforming growth factor beta (TGF beta), epidermal growth factor (EGF), platelet derived growth factor (PDGF), and/or cartilage derived growth factor (CDGF). As well, other biological components may be added to an expanded collagenous material, such as heparin, heparin sulfate, hyaluronic acid, fibronectin and the like. Thus, generally speaking, an expanded collagenous material may include a bioactive component that induces, directly or indirectly, a cellular response such as a change in cell morphology, proliferation, growth, protein or gene expression similar to a non-expanded collagenous material.

ECM tissue layers can be used in the manufacture of laminated graft body structures. For these purposes each bioresorbable sheet 200 can for example be comprised of about 1 to 10 ECM tissue layers. Illustratively, one bioresorbable sheet 200 may include only a single ECM tissue layer, and the other may include multiple (e.g. 1 to 10, or 2 to 6) ECM tissue layers. Sheets of multilaminate ECM tissue layers can be prepared in any suitable fashion. These include, for instance, laminating the layers together using dehydrothermal bonding under heated, non-heated or lyophilization conditions, using adhesives, glues or other bonding agents, stitching, crosslinking with chemical agents or radiation (including UV radiation), or any combination of these with each other or other suitable methods. For additional information as to techniques for laminating ECM layers to one another, reference may be made for example to U.S. Pat. Nos. 5,711,969, 5,755,791, 5,855,619, 5,955,110, 5,968,096, and to U.S. Patent Publication No. 20050049638.

All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

1. A device for closing a puncture in the wall of a blood vessel, comprising:
a first bioresorbable sheet;
a second bioresorbable sheet;
an anchor member comprising a shaft having a proximal and a distal end, said distal end supporting a plurality of radially disposed fingers configured to secure said first sheet against the interior vessel wall adjacent to the puncture; and
a locking member defining an opening receivable over said shaft and a plurality of radially disposed fingers extending outward from the opening and configured to secure said second sheet against an external vessel wall adjacent to the puncture.

2. The device of claim 1 wherein:
said fingers are generally cuneate

3. The device of claim 1 wherein:
said radially disposed fingers are independently flexible.

4. The device of claim 1 wherein:
said radially disposed fingers are concave with respect to the vessel wall.

5. The device of claim 1 wherein:
said anchor member and said locking member are composed of a resiliently deformable polymer.

6. The device of claim 1 wherein:
said anchor member and said locking member are composed of a bioresorbable material.

7. The device of claim 1 wherein:
said first sheet and said second sheet are composed of extracellular matrix material.

8. (canceled)

9. The device of claim 1 wherein:
said first sheet is affixed to said anchor member; and
said second sheet is affixed to said locking member.

10. The device of claim 1 further comprising:
a plurality of protrusions on said shaft, wherein said locking member is configured to attach to said protrusions.

11. The device of claim 1 wherein:
said shaft contains a compression limiting element comprising a portion of said shaft with a larger diameter than said opening.

12. (canceled)

13. The device of claim 1 further comprising:
a retention element, said retention element defining an elongation of said proximal end of said shaft.

14. The device of claim 1 further comprising:
a filament extending from said shaft.

15. A method for closing a puncture in the wall of a blood vessel, the method comprising:
inserting an anchor member and a first bioresorbable sheet through a puncture in a vessel wall, said anchor member comprising a shaft and a plurality of radially disposed fingers configured to secure said first sheet against the internal vessel wall surrounding the puncture; and
attaching a locking member with a second bioresorbable sheet to said shaft such that said locking member and said second sheet are positioned external to the punctured vessel and said shaft extends through the puncture, said locking member comprising a plurality of radially disposed fingers configured to secure said second sheet against the external vessel wall surrounding the puncture.

16. (canceled)

17. A device for closing a puncture in the wall of a body vessel, the device comprising:
an anchor member having a plurality of radially disposed fingers extending from a shaft, said anchor member self-expandable from a first position in which the support members are compressed for delivery through the puncture site, to a second position in which the support members extend transverse to the axis of said shaft; and
a locking member defining an opening receivable over said shaft and a plurality of radially disposed fingers extending radially outward from said opening, said opening configured for attachment to said shaft.

18. (canceled)

19. The device of claim 17 wherein:
said fingers are independently flexible.

20. (canceled)

21. The device of claim 17 wherein:
said anchor member and said locking member are composed of a resiliently deformable polymer.

22. (canceled)

23. The device of claim 17 further comprising:
a first bioresorbable sheet having a first opening, said first opening configured to fit over said shaft; and
a second bioresorbable sheet having a second opening, said second opening configured to fit over said shaft.

24. (canceled)

25. The device of claim 23 wherein:
said first sheet is affixed to said anchor member; and
said second sheet is affixed to said locking member.

26. The device of claim 17 further comprising:
a plurality of protrusions on said shaft, wherein said opening is configured to attach to said protrusions.

27. (canceled)

28. (canceled)

29. The device of claim 17 further comprising:
a filament extending from said shaft.
